(19) United States
(12) Patent Application Publication
(10) Pub. No.: US 2006/0241454 A1
(43) Pub. Date: Oct. 26, 2006

(54) Transmit multibeam for compounding ultrasound data

(75) Inventors: Kutay F. Ustuner, Mountain View, CA (US); Anming He Cai, San Jose, CA (US); Charles E. Bradley, Burlingame, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/099,866

(22) Filed: Apr. 5, 2005

(51) Int. Cl. A61B 8/00

(52) U.S. Cl. 600/447; 600/437

(57) ABSTRACT

Transmit multibeams insonify an object with multiple noncollinear transmit beams fired substantially simultaneously. The noncollinear beams are along different scan lines of same scan geometry, or they belong to scan lines of different scan geometries. One or more receive beams are formed in parallel in response to each of the noncollinear beams. The scan geometry and/or center frequency is varied between the noncollinear transmit beams of a transmit event. By scanning the transmit multibeam, and varying the scan geometry and/or frequency between the noncollinear transmit beams of a transmit event, multiple component images are generated for compounding. The component images are scan-converted (if scan geometries are different), weighted and combined after envelope detection.

### BACKGROUND

The present invention relates to compounding ultrasound data. In particular, spatial or frequency compounding is provided to increase detectability of specular targets and/or soft tissue inhomogeneities. Two or more component images are compounded after envelope detection. Each of the component images has a different spatial and/or temporal frequency response so that they are substantially uncorrelated. For example, scan lines associated with different angles relative to a transducer are used to form the two different component images. By summing these two component images (i.e., spatial compounding), the detectability of soft tissue lesions and anisotropic objects increases.

U.S. Pat. No. 6,790,181, the disclosure of which is incorporated herein by reference, discloses spatial compounding where the scan angles associated with each frame of data vary within the frame of data. The variation in angle allows for a fully overlapped scan region without pre-weighting or smoothing to account for regions with different numbers of component images.

Compounding with multiple component images reduces temporal resolution and may introduce temporal discordance artifacts. To increase frame rate, the spatial or temporal frequency response differences may be implemented using only receive parameter variations. However, using both transmit and receive parameter variations increases component image decorrelation, and therefore increases detectability of specular targets and soft tissue inhomogeneities. An alternative way to increase the compounding frame rate is to use receive multibeam with many parallel receive beams. But this requires widening the transmit beam which compromises detail and contrast resolution.

### BRIEF SUMMARY

The preferred embodiments described below include methods and systems for spatial and frequency compounding in ultrasound. A transmit multibeam insonifies an object with multiple noncollinear transmit beams fired substantially simultaneously. The noncollinear beams are along different scan lines of same scan geometry, or they belong to scan lines of different scan geometries. For example, a scan line set may constitute unsteered linear scan geometry or steered linear scan geometry. One or more receive beams are formed in parallel in response to each of the noncollinear beams. With different transmit beams with or without different frequencies for each transmit beam, data responsive to a same transmit event is used in different component images. Frequency variation between the transmit beams of a transmit event reduces inter-beam interference, thereby improving contrast resolution. The component images are scan-converted (if scan geometries are different), weighted and combined after envelope detection.

Due to spatial and/or frequency differences, the component images exhibit different spatial and/or temporal frequency responses. Therefore, they are sufficiently uncorrelated. Compounding of uncorrelated images increase detectability of specular targets and soft tissue inhomogeneities. Using simultaneous transmission and reception of beams increases frame rate and thus reduces temporal discordance between the component images of compounding. Distributing the parallel receive beams between the multiple noncollinear transmit beams of a transmit event limits the amount of transmit widening required to support a given number of parallel receive beams.

In a first aspect, a method is provided for increasing detectability of tissue inhomogeneities and specular targets in ultrasound. First and second noncollinear transmit beams are transmitted substantially simultaneously. The first transmit beam has a different angle, different center frequency or both different angle and center frequency than the second transmit beam. A first receive beam is received in response to the first transmit beam, and a second receive beam is received in response to the second transmit beam. A first frame of data including the first receive beam is compounded with a second frame of data including the second receive beam.

In a second aspect, a method is provided for increasing detectability of tissue inhomogeneities and specular targets in ultrasound. Two or more frames of data are formed with different angle or center frequency beam characteristics. The two or more frames of data are formed, at least in part, from data acquired in response to two or more non-collinear, substantially simultaneous transmit beams having the different angle or center frequency beam characteristics. The two or more frames of data are compounded.

In a third aspect, a system is provided for spatial and/or frequency compounding in ultrasound. A transmit beamformer is operable to transmit first and second noncollinear transmit beams substantially simultaneously. The first transmit beam has a different angle, different center frequency or different angle and center frequency than the second transmit beam. A receive beamformer is operable to receive a first receive beam in response to the first transmit beam and a second receive beam in response to the second transmit beam. A compound processor is operable to compound a first frame of data that includes the first receive beam with a second frame of data that includes the second receive beam.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for spatial and/or frequency compounding;

FIG. 2 is a graphical representation showing transmit and receive beam relationships with sets of data for spatial compounding in one embodiment; and

FIG. 3 is a graphical representation of one embodiment showing transit and receive beam relationships with sets of data for frequency compounding.

### DETAILED DESCRIPTION OF THE DRAWINGS AND PRESENTLY PREFERRED EMBODIMENTS

Transmit and receive compounding is performed with minimal temporal resolution loss and temporal discordance artifacts. Non-collinear transmit multi-beams are fired to insonify an object simultaneously along distinct transmit lines with distinct steering angles and/or center frequencies. Non-collinear receive multi-beams are formed to sample the object substantially along each of the transmit lines with substantially the same steering angle and/or related center frequency as the respective transmit beams. The non-collinear transmit beams and their respective receive beams for spatial compounding are on different master line grids. For example, one frame of data is formed using steered lines and another frame of data formed using unsteered lines. The distinct beam groups have different frame indices. For frequency compounding, the non-collinear transmit beams and their respective receive beams have different center frequencies, such as a low and high frequency. Spatial and frequency compounding may be provided together.

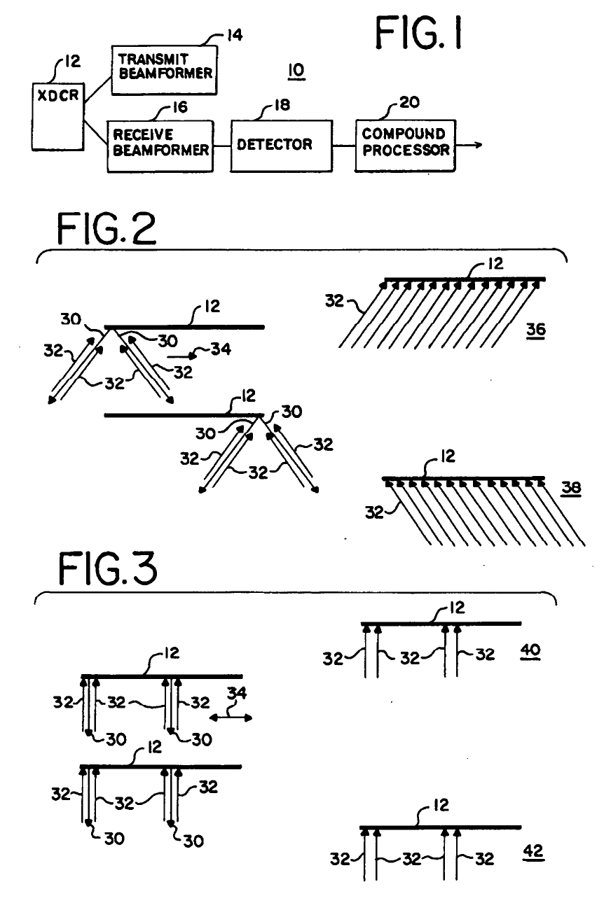

FIG. 1 shows one embodiment of a system 10 for reducing increasing detectability of tissue inhomogeneities and specular targets in ultrasound imaging. The system 10 is a medical diagnostic ultrasound imaging system, but other imaging systems using multiple transmit or receive antennas (i.e., elements) may be used. The system 10 implements spatial and/or frequency compounding using substantially simultaneous transmit of beams with different characteristics. The system 10 includes a transducer 12, a transmit beamformer 14, a receive beamformer 16, a detector 18 and a compound processor 20. Additional, different or fewer components may be provided, such as the system 10 without the detector 20 or with a scan converter and display device.

The transducer 12 is an array of a plurality of elements. The elements are piezoelectric or capacitive membrane elements. The array is configured as a one-dimensional array, a two-dimensional array, a 1.5 D array, a 1.25 D array, a 1.75 D array, an annular array, a multidimensional array, combinations thereof or any other now known or later developed array. The transducer elements transduce between acoustic and electric energies. The transducer 12 connects with the transmit beamformer 14 and the receive beamformer 16 through a transmit/receive switch, but separate connections may be used in other embodiments.

The transmit beamformer 14 is shown separate from the receive beamformer 16. Alternatively, the transmit and receive beamformers 14, 16 may be provided with some or all components in common. Operating together or alone, the transmit and receive beamformers 14, 16 form beams of acoustic energy for scanning a one, two or three dimensional region. Vector®, sector, linear or other scan formats may be used. A single receive beam is generated for each transmit beam. Alternatively, two or more receive beams are generated for each transmit beam. Data representing scan lines may be synthesized from coherent receive beam data, such as disclosed in U.S. Pat. No. 5,623,928, the disclosure of which is incorporated herein by reference. Fully populated control data sets for any of the transmit or receive beamformer parameters discussed herein are provided. Alternatively, sparse sets are used for real-time calculation of the control data, such as disclosed in U.S. Pat. No. 5,581,517, the disclosure of which is incorporated herein by reference.

The transmit beamformer 14 is a processor, delay, filter, waveform generator, memory, phase rotator, digital-to-analog converter, amplifier, combinations thereof or any other now known or later developed transmit beamformer components. In one embodiment, the transmit beamformer 14 is the transmit beamformer disclosed in U.S. Pat. No. 5,675,554, the disclosure of which is incorporated herein by reference. The transmit beamformer 14 digitally generates envelope samples. Using filtering, delays, phase rotation, digital-to-analog conversion and amplification, the desired transmit waveform is generated. Other waveform generators may be used, such as switching pulsers or waveform memories.

The transmit beamformer 14 is configured as a plurality of channels for generating electrical signals of a transmit waveform for each element of a transmit aperture on the transducer 12. The waveforms are unipolar, bipolar, stepped, sinusoidal or other waveforms of a desired center frequency or frequency band with one, multiple or fractional number of cycles. The waveforms have relative delay and/or phasing and amplitude for focusing the acoustic energy. The transmit beamformer 14 includes a controller for altering an aperture (e.g. the number of active elements), an apodization profile (e.g., type or center of mass) across the plurality of channels, a delay profile across the plurality of channels, a phase profile across the plurality of channels, center frequency, frequency band, waveform shape, number of cycles and combinations thereof. A scan line focus is generated based on these beamforming parameters.

The transmit beamformer 14 is operable to transmit at least two transmit beams of ultrasound energy substantially simultaneously. Substantially simultaneously accounts for differences in steering direction or depth of focus and associated time of transmission from any given element of the transducer 12. Separately generated waveforms are summed together for each channel, or a complex waveform representing the relatively delayed and apodized waveforms for two beams is generated for each channel. The transmit beams are generated for a same transmit event, but have different angles, different center frequencies or both different angles and center frequencies. For example, transmit beams with angles of greater than 5, 10, 15, 20, 45, 90 or other degree difference from each other with or without the same origin on the transducer 12 are generated. In one embodiment, two transmit beams with about 70 degree difference (each 35 degrees from normal) are generated in a same transmit event. In another embodiment, three transmit beams with about 20 degree difference (one normal and the other two at 20 degrees from normal) are generated in a same transmit event. As another example, transmit beams with center frequency differences in the Kilohertz or Megahertz ranges are provided. In one embodiment, the center frequencies are different by about ¼ of the center frequency of the highest center frequency (e.g., 3 and 4 MHz).

The transmit beams formed at a substantially same time are non-collinear. The transmit beams have different angles and/or different origins. The differences result in insonifying different regions of the patient along at least a portion of the beams. The different regions are relative to an imaging resolution provided by the system 10.

The receive beamformer 16 is a preamplifier, filter, phase rotator, delay, summer, base band filter, processor, buffers, memory, combinations thereof or other now known or later developed receive beamformer components. In one embodiment, the receive beamformer is one disclosed in U.S. Pat. Nos. 5,555,534, 5,921,932 and 5,685,308, the disclosures of which are incorporated herein by reference. Other analog or digital receive beamformers capable of receiving two or more beams in response to a transmit event may be used.

The receive beamformer 16 is configured into a plurality of channels for receiving electrical signals representing echoes or acoustic energy impinging on the transducer 12. A channel from each of the elements of the receive aperture within the transducer 12 connects to an amplifier and/or delay for applying apodization amplification. An analog-to-digital converter digitizes the amplified echo signal. The digital radio frequency received data is demodulated to a base band frequency. Any receive delays, such as dynamic receive delays, and/or phase rotations are then applied by the amplifier and/or delay. A digital or analog summer combines data from different channels of the receive aperture to form one or a plurality of receive beams. The summer is a single summer or cascaded summer. The summer sums the relatively delayed and apodized channel information together to form a beam. In one embodiment, the beamform summer is operable to sum in-phase and quadrature channel data in a complex manner such that phase information is maintained for the formed beam. Alternatively, the beamform summer sums data amplitudes or intensities without maintaining the phase information.

Beamforming parameters including a receive aperture (e.g., the number of elements and which elements used for receive processing), the apodization profile, a delay profile, a phase profile, imaging frequency and combinations thereof are applied to the receive signals for receive beamforming. For example, relative delays and amplitudes or apodization focus the acoustic energy along one or more scan lines. A control processor controls the various beamforming parameters for receive beamformation. The values provided for the beamformer parameters for the receive beamformer 16 are the same or different than the transmit beamformer 14. For example, an aberration or clutter correction applied for receive beam formation is different than an aberration correction provided for transmit beam formation due to differences in signal amplitude.

The receive beamformer 16 is operable to form receive beams in response to the transmit beams. For example, the receive beamformer 16 receives one or two receive beams in response to each transmit beam. The receive beams are collinear, parallel and offset or nonparallel with the corresponding transmit beams. Since two or more transmit beams are substantially simultaneously formed, two or more responsive receive beams are substantially simultaneously formed in a same receive event. Alternatively, the data received at the elements of the receive aperture in a receive event is stored for sequentially forming the two or more receive beams in response to the same transmit event.

The receive beamformer 16 outputs image data, data representing different spatial locations of a scanned region. The image data is coherent (i.e., maintained phase information), but may include incoherent data. The data may be formed by processing received data, such as synthesizing scan lines (i.e., coherent combination), or other processes for generating data used to form an image from received information. For example, inter-beam phase correction is applied to one or more beams, and then the phase corrected beams are combined through a coherent (i.e., phase sensitive) filter to form synthesized ultrasound lines and/or interpolated between beams to form new ultrasound lines. Once the channel data is beamformed or otherwise combined to represent spacial locations of the scanned region, the data is converted from the channel domain to the image data domain.

The detector 18 is a general processor, digital signal processor, application-specific integrated circuit, control processor, digital circuit, summer, filter, finite impulse response processor, multipliers, field programmable gate array, combinations thereof or other now known or later developed processors for forming incoherent image data from received signals. The detector 18 includes a single or multiple processors with or without log compression. The detector 18 detects the amplitude, intensity, log-compressed amplitude or power of the beamformed signals. For example, the detector 18 is a B-mode detector of the signal envelope. One or more filters, such as spatial or temporal filters may be provided with the detector 18. The detector 18 outputs incoherent image data. Additional processes, such as filtering, interpolation, and/or scan conversion, may be provided by the detector 18 or other component before the compounding processor 20.

The compounding processor 20 is a general processor, control processor, digital signal processor, application specific integrated circuit, field programmable gate array, analog circuit, digital circuit, summer, combinations thereof or other now known or later developed device for compounding. The compounding processor 20 is operable to compound two or more frames of data. Data representing the same or similar spatial locations from the two or more frames of data are averaged. Weighted averaging, selection of the maximum or other functions are alternatively used. Low pass filtering or other processes may be used to remove any artifact from compounding frames of data where the number of frames compounded varies within a scanned region. Where the scan geometries for each frame of data are different, scan conversion may be used prior to compounding.

Where the frames of data have different center frequency characteristics, frequency compounding is provided. Where the frames of data have different scan angles, angle combinations or scan geometry, spatial frequency compounding is provided. Both spatial and frequency compounding may be used. By forming the frames of data from transmit beams transmitted substantially simultaneously, temporal resolution is maintained. By using non-collinear transmit beams, multiple frames of data with different characteristics are formed at substantially the same time, further limiting temporal discordance artifacts.

FIGS. 2 and 3 show two different embodiments of compounding to increase detectability of tissue inhomogeneities and specular targets in ultrasound. FIG. 2 shows transmit beams 30 and receive beams 32 using different angles to acquire two different data frames 36, 38 for spatial compounding. FIG. 3 shows transmit beams 30 and receive beams 32 for forming two different frames of data 40, 42 for frequency compounding. The frames of data 36 and 38 of FIG. 2 are associated with scan lines with different angle beam characteristics. The receive beams 32 of the frame of data 36 are at an equal or non-equal angle on an opposite side of a normal line to the transducer 12 as the receive lines 32 of the other frame of data 38. Additional, different or fewer frames of data may be provided. Similarly, the frames of data 40 and 42 shown in FIG. 3 show incomplete frames. Each frame of data 40 and 42 corresponds to different center frequency beam characteristics. The receive beams 32 are at a same angle relative to the transducer 12. In yet other embodiments, both frequency and spatial compounding are provided, such as acquiring the frame of data 36 of FIG. 2 with a different center frequency of each of the receive beams 32 as well as a different angle as compared to the frame of data 38 and the corresponding receive beams 32.

FIG. 2 shows two different transmit and associated receive events. In the first, two transmit beams 30 of ultrasound energy are generated substantially simultaneously. The two transmit beams 30 have a same origin, but may have different origins. The transmit beams are non-collinear. Similarly, FIG. 3 shows two different transmit events and corresponding receive events. Two transmit beams 30 of ultrasound energy are transmitted substantially simultaneously from the transducer 12. In the upper transmit event, the transmit beams have different center frequencies, such as the left most transmit beam 30 having a 3 megahertz frequency and a right most transmit beam 30 having a 4 megahertz frequency. In a later occurring transmit event, the opposite center frequencies are assigned to the same transmit beams 30. Three or more transmit beams may be generated substantially simultaneously.

Other characteristics of the transmit beams 30 may vary as a function of the transmit beam 30. For example, the different transmit beams have different temporal frequency response. A different center frequency, band width or frequency response shape of a transmit beam 30 relative to another transmit beam 30 in a same transmit event is provided. As another example, different focal depths are provided. As yet another example, a different aperture size, apodization type (e.g. Gaussian and an equally weighted apodization profile), apodization center of mass (e.g. center of the aperture and off-set from the center of the aperture) or combinations thereof. As yet other example, the transmit beams 30 transmitted at substantially the same time have complimentary codes. Spread spectrum, phase shift keying, chirp or other now known or later developed coding may be used. The receive beams 32 are decoded. The coding provides for additional rejection between spatially distinct scan lines.

In response to a given transmit event, one or more receive beams 32 are formed for each of the transmit beams 30. In the examples shown in FIGS. 2 and 3, two receive beams 32 are formed in response to each of the transmit beams 30 in a given transmit and receive event. The two receive beams 32 are parallel but non-collinear with the transmit beam 30. In an alternative embodiment, a receive beam 32 is co-linear with the transmit beam 30. Different numbers of receive beams 32 may be formed for different ones of the substantially simultaneously generated transmit beams 30.

The receive beams 32 are formed using the same or different beamforming parameters as the transmit beams 30. Other than receiving along spatially different scan lines shown in FIGS. 2 and 3, another example is receiving at a different frequency. For example, the receive beams 32 are associated with a harmonic or sub-harmonic of the corresponding transmit beams 30. A harmonic includes integer as wells as fractional harmonics. Where different center frequencies are provided for the transmit beams 30, the harmonic or sub-harmonics for the receive beams 32 are similarly different. In alternative embodiments, the transmit or receive center frequencies are the same but with different receive or transmit center frequencies, respectively.

The beamforming parameters for received beams 32 formed in response to a same transmit event may similarly be the same or different. For example, receive beams 32 formed in response to different transmit beams 30 have different aperture size, apodization type, apodization center of mass or combinations thereof. The receive beams 32 formed in response to a same transmit beam may have the same or different beamform parameters other than focusing along different scan lines adjacent to the transmit beam 30.

The transmission of multiple transmit beams 30 substantially simultaneously and the associated received events are repeated along at least a lateral dimension 34. In the example of FIG. 2, the origin shared by the transmit beams 30 is translated across the transducer 12 for each repetition. As a result, the received beams 32 shown for the data frames 36 and 38 are acquired. In the example of FIG. 3, the same or different scan lines are used for each of the frames 40, 42 to provide frames of data representing a plurality of scan lines with different frequency characteristics. Data representing a two or three dimensional region is acquired, such as varying the transmission and reception events along two lateral dimensions for three dimensional scanning.

Other than focusing along different scan lines, the transmit and/or receive beamforming parameters may vary as a function of lateral position or scanning. For example, the angle or scan pattern varies throughout a two or three dimensional region as disclosed in U.S. Pat. No. 6,790,181, the disclosure of which is incorporated herein by reference. As another example, scan lines associated with greater angles away from normal to the transducer 12 have a lower frequency, such as disclosed in U.S. Pat. No. 5,549,111, the disclosure of which is incorporated herein by reference. The center frequency used for a given transmit and receive beam 30, 32 is a function of the element spacing, effective element width and the angle away from the normal. For frequency compounding, the center frequency varies as a function of angle within a given frame of data 40, 42. Similar variation is provided for each of the frames 40, 42 such that a frequency difference is provided for any given spatial location represented by both frames of data 40, 42.

After a series of transmit and associated receive events, each frame of data 36, 38 or 40, 42 is formed. Received beams 32 represented in each frame 36, 38, 40, 42 are formed as part of a same receive events as receive beams 32 in other sets of data 38, 36 or 42, 40. Each frame of data represents an at least overlapping spatial region, such as a two or three dimensional region. The data for each of the frames of data 36, 38 or 40, 42 are detected after beam formation and prior to compounding. For example, envelope or B-mode detection is provided. Alternatively, harmonic, Doppler or another form of detection is provided. In yet other alternative embodiments, the detection is performed after combining the sets of data 36, 38 or 40, 42. For example, data synthesis is provided.

After two or more frames of data associated with different angle and/or center frequency characteristics are formed, the frames of data are compounded. For data from different frames representing a same spatial location, the data is averaged. If the data represents adjacent but different spatial locations, interpolation converts the data in different frames of data to a same grid for compounding. Alternatively, weighted averaging is provided. Spatial regions associated with a single frame of data may be discarded or included in a compound frame. Low pass filtering or other techniques may be used to remove or minimize compounding artifacts. Since each frame of data includes received beams 32 from the same transmit events, the spatial or frequency compounding with limited temporal discordance artifacts is provided.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for increasing detectability of tissue inhomogeneities and specular targets in ultrasound, the method comprising:
transmitting first and second noncollinear transmit beams substantially simultaneously, the first transmit beam having a different angle, different center frequency or both different angle and center frequency than the second transmit beam;
receiving a first receive beam in response to the first transmit beam and a second receive beam in response to the second transmit beam;
compounding (i) a first frame of data including the first receive beam with (ii) a second frame of data including the second receive beam.

2. The method of claim 1 further comprising repeating the transmitting and receiving along at least a lateral dimension, the first and second frames of data including data from each repetition such that at least a two dimensional area is scanned.

3. The method of claim 1 wherein transmitting comprises transmitting with the different center frequency.

4. The method of claim 1 wherein transmitting comprises transmitting with the first transmit beam having a different temporal frequency response than the second transmit beam.

5. The method of claim 1 wherein transmitting comprises transmitting with the different angle.

6. The method of claim 1 wherein transmitting comprises transmitting with the first transmit beam having a different focal depth than the second transmit beam.

7. The method of claim 1 wherein transmitting comprises transmitting with the first transmit beam having a different aperture size, apodization type, apodization center of mass or combinations thereof than the second transmit beam.

8. The method of claim 1 wherein transmitting comprises transmitting with the first and second transmit beams having complementary codes.

9. The method of claim 1 further comprising detecting before compounding;
wherein compounding comprises averaging the first frame of data with the second frame of data for overlapping regions.

10. The method of claim 1 wherein receiving comprises receiving the first and second receive beams at a harmonic or sub-harmonic of the first and second transmit beams, respectively.

11. The method of claim 1 wherein receiving comprises receiving the first receive beam with a different aperture size, apodization type, apodization center of mass or combinations thereof than the second receive beam.

12. The method of claim 1 wherein receiving comprises receiving the first and second receive beams with first and second frequencies respectively,

13. The method of claim 12 wherein the first and second frequencies are a function of element spacing, effective element width and the different angle.

14. A method for increasing detectability of tissue inhomogeneities and specular targets in ultrasound, the method comprising:
forming two or more frames of data with different angle or center frequency beam characteristics, at least in part, from data acquired in response to two or more non-collinear, substantially simultaneous transmit beams having the different angle or center frequency beam characteristics; and
compounding the two or more frames of data.

15. The method of claim 14 further comprising repeating the forming along at least a lateral dimension, each of the two or more frames of data including data from each repetition.

16. The method of claim 14 wherein forming comprises forming the different center frequency beam characteristic.

17. The method of claim 14 wherein forming comprises forming with the different angle beam characteristic.

18. The method of claim 14 wherein forming comprises forming with a different temporal frequency response, a different focal depth, a different aperture size, apodization type, apodization center of mass, complementary codes, and combinations thereof for each of the two or more frames of data.

19. A system for spatial and/or frequency compounding in ultrasound, the system comprising:
a transmit beamformer operable to transmit first and second non-collinear transmit beams of ultrasound energy substantially simultaneously, the first transmit beam having a different angle, different center frequency or both different angle and center frequency than the second transmit beam;
a receive beamformer operable to receive a first receive beam in response to the first transmit beam and a second receive beam in response to the second transmit beam; and
a compounding processor operable to compound a first frame of data including the first receive beam with a second frame of data including the second receive beam.

20. The system of claim 19 wherein the transmit beamformer is operable to transmit the first transmit beam with the different center frequency than the second transmit beam.

21. The system of claim 19 wherein the transmit beamformer is operable to transmit the first transmit beam with the different angle than the second transmit beam.

22. The system of claim 19 further comprising:
a detector operable to envelope detect an output from the receive beamformer, the detector outputting the first and second frames of data to the compounding processor.
